(19) United States
(12) Patent Application Publication
(10) Pub. No.: US 2015/0335441 A1
(43) Pub. Date: Nov. 26, 2015

(54) MULTI-COMPONENT SHOULDER IMPLANT ASSEMBLY WITH DUAL ARTICULATING SURFACES

(71) Applicant: Linares Medical Devices, LLC, Auburn Hills, MI (US)

(72) Inventors: Miguel A. Linares, Bloomfield Hills, MI (US); Miguel A. Linares, JR., Bloomfield Hills, MI (US)

(21) Appl. No.: 14/815,530

(22) Filed: Jul. 31, 2015

Related U.S. Application Data
(63) Continuation-in-part of application No. 14/258,492, filed on Apr. 22, 2014.
Application No. 14/258,492 is a division of application No. 13/592,738, filed on Aug. 23, 2012, now Pat. No. 8,702,800.
(60) Provisional application No. 61/526,388, filed on Aug. 23, 2011.
(60) Provisional application No. 61/526,404, filed on Aug. 23, 2011.

(51) Int. Cl. A61F 2/40

(52) U.S. Cl. CPC A61F 2/4014; A61F 2/4003; A61F 2/4081; A61F 2002/4007; A61F 2002/4085; A61F 2220/0008; A61F 2230/0063

(57) ABSTRACT

An implant assembly for re-establishing a glenohumeral joint between scapula and humerus bones. A ball has a first exposed convex support surface and a first reverse convex bone contacting surface extending from a first annular perimeter edge established with the first convex support surface. A first stem extends from the first reverse convex surface such that the first reverse convex surface and first stem are adapted to being mounted into a reconditioned glenoid cavity defined in the scapula. A receiver has a second exposed convex surface, a rim edge extending about a portion of said second exposed convex surface and defining a concave profile recessed within the second exposed convex surface and so that the concave profile seats a portion of the first exposed convex surface of the ball in an articulating relationship. The receiver further has a second reverse convex bone contacting surface extending from a second annular perimeter established with the second exposed convex surface. A second stem extends from the second reverse convex surface such that the second reverse convex surface and the second stem are adapted to being mounted to a reconditioned humeral head associated with the humerus.

### CROSS-REFERENCE TO RELATED APPLICATIONS

This Application is a Continuation-in-part of application Ser. No. 14/258,492 filed on Apr. 22, 2014. Application Ser. No. 14/258,492 is a Division of application Ser. No. 13/592,738 filed on Aug. 23, 2012. Application Ser. No. 13/592,738 claims the benefit of U.S. Provisional Application 61/526,388 filed on Aug. 23, 2011. Application Ser. No. 13/592,738 claims the benefit of U.S. Provisional Application 61/526,404 filed on Aug. 23, 2011, the contents of which are incorporated herein in their entirety.

### FIELD OF THE INVENTION

The present invention is directed to a shoulder implant assembly and, more specifically, to a multi-component implant assembly incorporating a ball and a receiver mounted to first and second shoulder joint defining bones. An optional third substantially spherical shaped and intermediate defining component establishes dual and spaced apart universal and articulating surfaces with the fixedly mounted ball and receiver providing evenly distributed wear profiles for increased useful life of the implant, as well as relieving associated ligament tension.

### BACKGROUND OF THE INVENTION

The prior art discloses various types of artificial implants, such as replacing damaged natural joint constructions including those for the shoulder. Examples of these include each of the modular humeral head resurfacing system of Winslow et al., US 2006/0009852 and US 2005/0107882, each of which incorporates a two piece humeral component for use in joint arthroplasty which is adapted to be implanted into a joint and engaged by a likewise implanted socket component.

Isch et al, US 2010/0049327, teaches a system for replacing a portion of a shoulder joint including a tray having an outer surface and an inner surface, the outer surface adapted to engage a humeral stem. A bearing includes an outer bearing surface and an inner articulating surface, the bearing surface being positioned adjacent to the tray.

A ring is selectively disposed between the tray and the bearing and defines an annular body having a slit formed therein. The ring is movable between a locked position wherein the ring retains the bearing relative to the tray and an unlocked position. A removal tool defines a proximal end and a distal end. The distal end defines a ring engaging surface and a bearing engaging surface. The ring engaging surface is adapted to engage the ring at the slit such that the ring expands radially from the locked position away from engagement with the bearing to the unlocked position. The bearing engaging surface is adapted to engage the bearing and urge the bearing away from the tray.

US 2006/0020344, to Schultz et al., teaches a shoulder implant assembly and associated method for selectively performing reverse and traditional arthroplasty for a shoulder joint that includes a humerus and a glenoid. The implant assembly includes a head, a cup, a humeral stem and an adaptor. The method includes inserting the humeral stem to the humerus and connecting a male taper of the adaptor to a female taper of the head. For reverse arthroplasty, the method includes attaching the adaptor to the glenoid and the cup to the stem. For traditional arthroplasty, the method includes attaching the adaptor to the humeral stem and the cup to the glenoid. The method also includes articulating the head with the cup.

### SUMMARY OF THE INVENTION

The present invention discloses an implant assembly for re-establishing a glenohumeral joint between a scapular and humerus. The implant includes a ball having a first exposed convex support surface. A first reverse convex bone contacting surface extends from a first annular perimeter edge established with the first convex support surface. A first stem extends from the first reverse convex surface such that the first reverse convex surface and first stem are adapted to being mounted into a reconditioned glenoid cavity defined in the scapula.

A receiver has a second exposed convex surface, a rim edge extending about a portion of said second exposed convex surface and defining a concave profile recessed within the second exposed convex surface and so that the concave profile seats a portion of the first exposed convex surface of the ball in an articulating relationship. The receiver further has a second reverse convex bone contacting surface extending from a second annular perimeter established with the second exposed convex surface. A second stem extends from the second reverse convex surface such that the second reverse convex surface and the second stem are adapted to being mounted to a reconditioned humeral head associated with the humerus.

A first axis extending through a central location of the concave profile in the receiver and defines a non collinear angle relative to a second axis extending through a centerline of the second stem. The receiver further has a first interior component extending between an exposed surface of the concave profile which is intersected by a plane passing through the second annular perimeter, a dimension of the first interior component being greater than that of a second interior component extending beyond the first component to the second reverse convex bone contacting surface and in order to space the humerus from the scapula so as to maximize articulating motion between said ball and receiver.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is an assembled view of a yet further modified shoulder implant assembly exhibiting only first and second scapula and humerus mounted components and eliminating the inter-disposed and supported spheroid shaped component; and

FIG. 2 is an exploded view of the arrangement of FIG. 1 and better depicting the inner concavity profile defined in the humerus mounting receiver and for seating the ball mounted in the scapula;

FIG. 2A is an enlarged perspective of the receiver component which better illustrates the dimensioning established between the exposed convex surface and the reverse bone contacting surface and which serves to displace away from the humeral head the articulating interface between the ball exposed convex surface and the concave profile defined within the exposed convex surface of the receiver; and

FIGS. 3-5 depict a series of supporting Prior Art illustrations of a human anatomical shoulder joint, and for which the multi-component assembly provides an in situ and reconditioned implantation option.

### DETAILED DESCRIPTION OF THE PREFERRED EMBODIMENTS

As will be described in additional detail with reference to the succeeding variants, the present invention discloses a multi-component shoulder implant assembly for providing an in-situ and reconditioned installation option which is an improvement over other conventional joint implant installations.

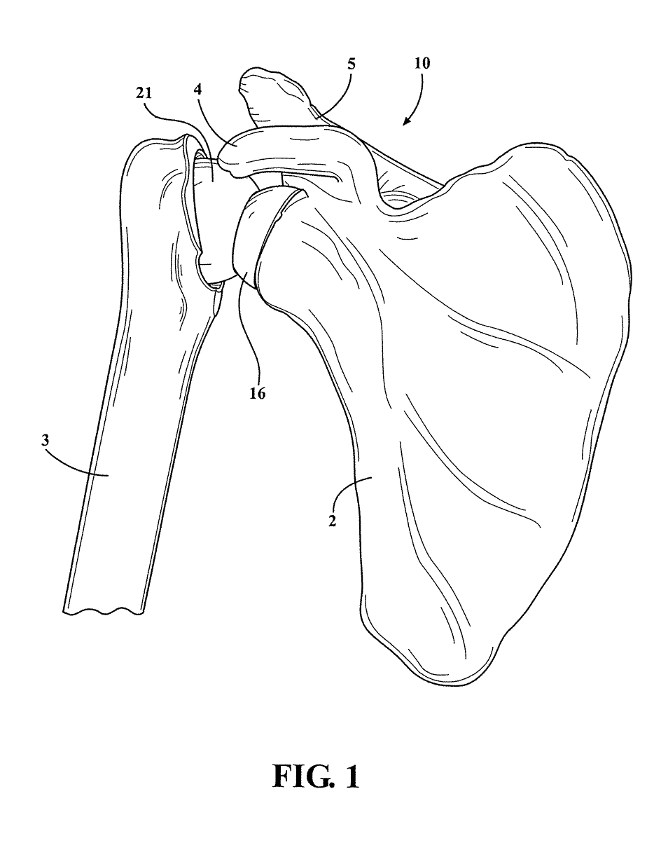

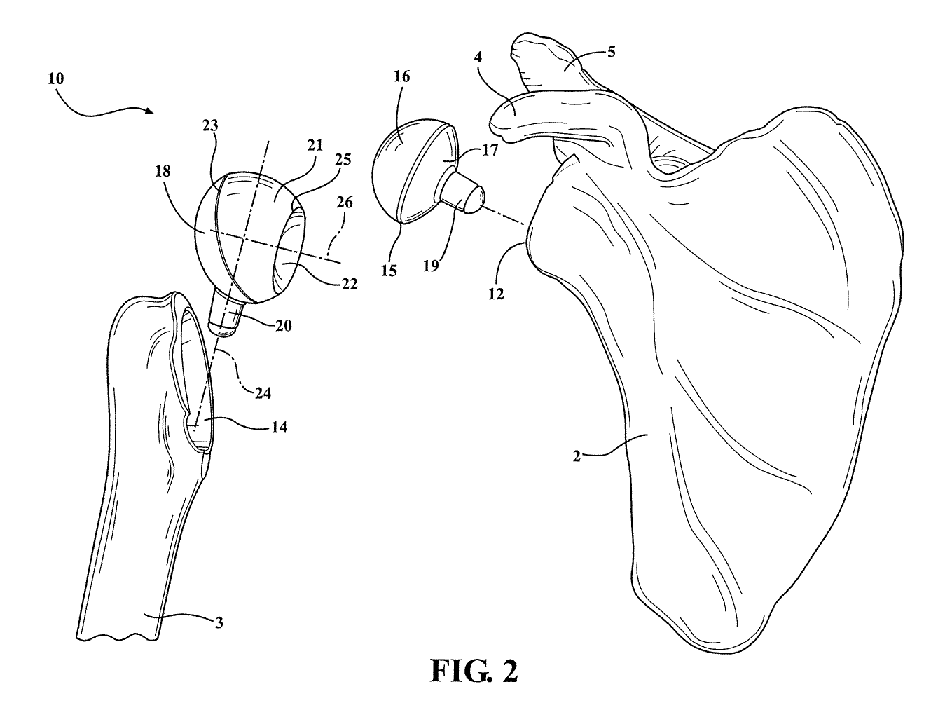

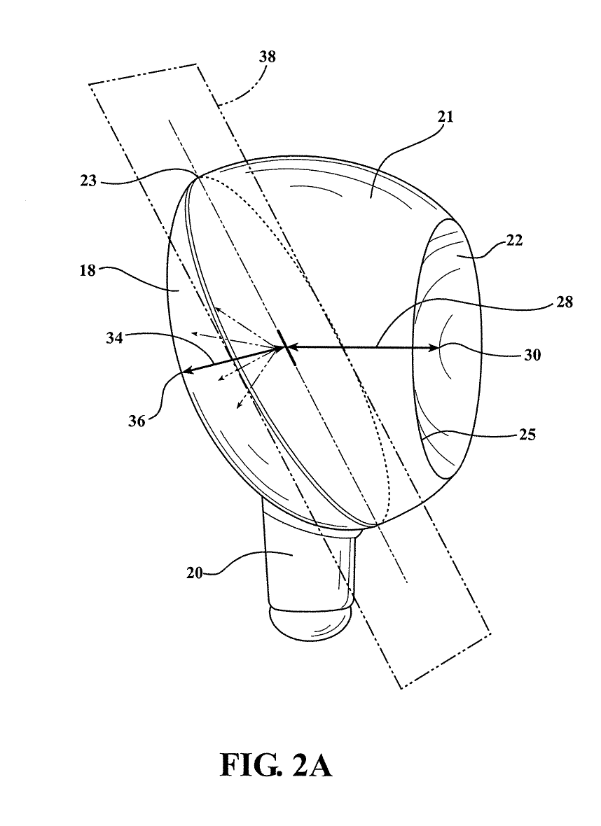

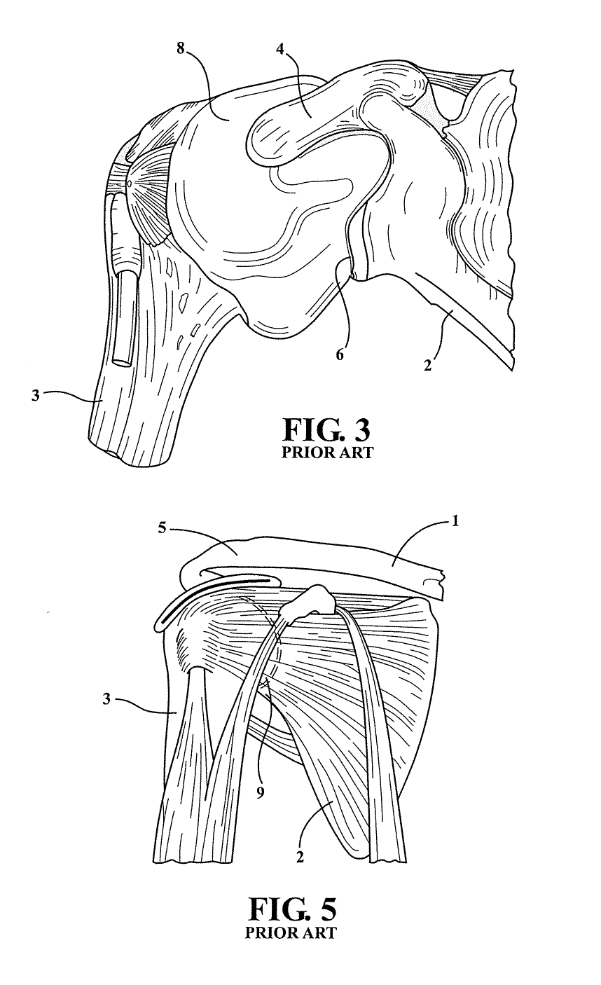

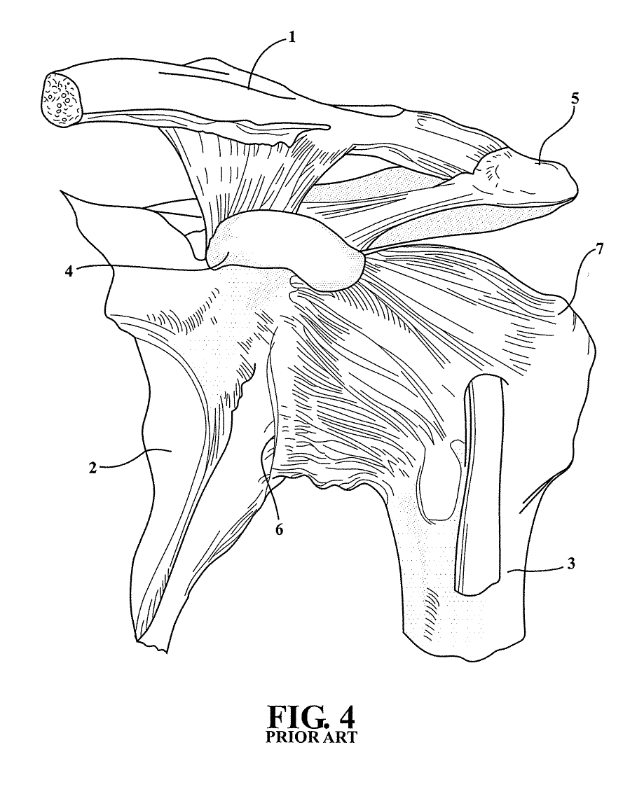

Prior to describing in detail the configuration of the multi-component shoulder implant depicted in FIGS. 1-2, a Prior Art depiction of an anatomically correct human shoulder is shown in FIGS. 3-5 and which includes three bones consisting of the clavicle (collarbone) 1, the scapula (shoulder blade) 2, and the humerus (upper arm bone) 3, as well as associated muscles, ligaments and tendons (see in particular FIGS. 4 and 5). The articulations between the bones of the shoulder collectively make up the shoulder joints where the humerus 3 attaches to the scapula 2.

An abbreviated and incomplete description of the scapula further includes, at strategic locations a coracoid process 4 and spine connected acromion 5, in the proximity of which is configured the glenoid cavity 6. The humerus 3 terminates, in relevant part, at an upper end located humeral head 7 (FIG. 4) which generally seats via an interposed bursa 8 (FIG. 3).

The three joints of the shoulder further include each of the glenohumeral, acromioclavicular and sternoclavicular joints. The glenohumeral joint, see as identified at 9 in FIG. 5, is the main joint of the shoulder and the generic term “shoulder joint” usually refers to this ball and socket joint that allows the arm to rotate in a circular fashion or to hinge out and up away from the body.

As is best depicted in the prior art view of FIG. 4, associated types of joint cartilage include articular cartilage located on the ends of the bones and which allows the bones to glide and move on each other and labrum cartilage located in the shoulder. In combination, the shoulder as constructed exhibits sufficient mobile for undertaking a wide range of actions of the arms and hands as well as being sufficiently stable as to allow for actions such as lifting, pushing and pulling. This compromise between mobility and stability results in a large number of shoulder problems not faced by other joints such as the hip.

With reference now to the embodiments of the invention set forth in FIGS. 1-2, and for purposes of ease and clarity of illustration, a simplified depiction is shown of the glenohumeral joint established between the scapula 2 and humerus 3 and in which all ligaments, muscles and tendons are removed. In each instance, and prior to installation of the multi-component implant assembly (such as occurring after significant degradation of the natural glenohumeral joint or in other instances in which an accident or other traumatic incident has resulted in significant damage), an initial (in situ) surgical reconditioning procedure is employed of the opposing joint defining surfaces established by the humeral head 7 and the glenoid cavity 6. This includes employing relevant surgical drilling and shaping instruments (also not shown) in order to prepare the joint defining locations of the bones for subsequently attaching selected components associated with the implant assembly and as will now be described.

The above stated, and referring initially to each of FIGS. 1 and 2, a pair of assembled and exploded views, both generally at 10, are depicted of a shoulder implant assembly for installation within reconditioned and opposing end locations of the patient's scapula 2 (represented by reconditioned profile 12) and humerus (further represented by reconditioned profile 14), and as is best shown in the exploded view of FIG. 2. The implant assembly 10 includes, collectively, a stem supported and substantially semi-spherical component, also termed a ball element having a main convex surface 16 defining an annular perimeter with a reverse convex bone contacting surface 17 which is mounted within the reconditioned recess 12 of the scapula glenoid cavity. The main convex 16 and reverse bone contacting 17 surfaces are separated by a first annular perimeter edge 15 established therebetween. A first stem 19 extends from the reverse convex bone contacting surface 17 and is adapted to being mounted within the reconditioned glenoid cavity 12 defined in the scapula 2.

A further receiver component includes a reverse convex bone contacting surface (see at 18), a stem mounting portion 20 projecting from a lower end location of the bone contacting surface 18 and which is adapted to being mounted to the reconditioned humeral head 14 of the humerus 3. The receiver component exhibits a substantially spherical shape with a second exposed convex surface (see at 21 and which likewise defines an annular or perimeter boundary 23 with the convex bone contacting surface 18).

A concave profile 22 is configured at an outer exposed seating end of the exposed convex surface 21 of the receiver component, the concave profile 22 exhibiting a rim edge 25 extending about a portion of the second exposed convex surface 21 and which is sized for seating directly the convex surface 16 of the ball element (see again FIG. 2) in order to define an articulating relationship between the ball and receiver. As further shown in FIG. 2, an axis 24 extending through a center location of the stem mounting portion 20 extends at a non collinear angle relative to a further axis 26 extending through the receiver component at a midpoint location of the concave profile 22 (such as in particular depicted as a substantially right angle).

The ball and receiver are each constructed of any suitable material including any type of plastic, metal or admixed composite. While not limited to any specific variant, the material selection for these components can alternate between any suitable material (e.g. heavy duty, wear resistant and sanitary polymeric, polymeric composite, surgical steel/aluminum, other metal or metal composite, as well as plastic/metal admixture. In a typical surgical procedure, a medical bonding cement or other suitable fastener/adhesive (not shown) is employed for anchoring the ball and receiver to the respective bone 2 and 3 ends, it being further understood that the configuration of these elements is capable of being reversed (e.g. the ball mounting to the humeral head and the receiver to the scapula glenoid cavity).

Referring now to FIG. 2A, an enlarged perspective is shown of the receiver and which better illustrates the dimensioning established between the exposed convex surface 21 and the reverse bone contacting surface 18, and which serves to displace away from the humeral head the articulating interface between the ball exposed convex surface 16 and the concave profile 22 defined within the exposed convex surface 21 of the receiver. As shown, the receiver further has a first interior dimension (or distance component 28) extending between an exposed surface (see non-limiting location 30) of the concave profile 22, which is intersected by a plane 32 passing through the second annular perimeter 23.

The first interior dimension 28 (taken at any location within the concave profile 22 up to the outer rim edge 25) is greater at every three dimensional surface location within profile 22 than that of a second interior dimension (or distance component 34) extending beyond an intersecting location 35 of the first component 30 with the intersecting plane 22, to any exterior location (such as depicted in non-limiting example at 36) extending along the three dimensional profile of the second reverse convex bone contacting surface 18. In this fashion, the construction of the receiver enables the implantable joint to space the humerus 3 from the scapula 2 so as to maximize articulating motion (see again FIG. 1) between the convex exposed surface 16 of the ball and the concave pocket 22 in the receiver.

Referring again to the existing arrangement of ligaments, tendons and muscles depicted in the Prior Art views of FIGS. 3-5, these provide the anchoring/seating support for retaining the articulating relationships established between the ball element 16 and receiver component 18, it further being understood that the components are capable of being retrofit installed within the reconditioned bone ends of the patient without the necessity of the ligaments and tendons being severed of otherwise impacted, thereby enhancing the universal motion profile afforded by the design and likewise reducing recovery time for the patient. As previously described, the ability to segment the articulating motion of the gleno-humeral joint into a pair of spaced articulating surfaces serves to both enhance artificial joint mobility as well as to more evenly distribute an associated wear profile of the joint, thereby increasing expected life of the assembly.

Having now described our invention, other and additional preferred embodiments will become evident to those skilled in the art to which it pertains, and without deviating from the scope of the appended claims.

We claim:

1. An implant assembly for re-establishing a glenohumeral joint between a scapula and humerus, comprising:
a ball having a first exposed convex support surface, a first reverse convex bone contacting surface extending from a first annular perimeter edge established with said first convex support surface, a first stem extending from said first reverse convex surface such that said first reverse convex surface and first stem are adapted to being mounted into a reconditioned glenoid cavity defined in the scapula;
a receiver having a second exposed convex surface, a rim edge extending about a portion of said second exposed convex surface and defining a concave profile recessed within said second exposed convex surface, said concave profile seating a portion of said first exposed convex surface of said ball in an articulating relationship, said receiver further having a second reverse convex bone contacting surface extending from a second annular perimeter established with said second exposed convex surface, a second stem extending from said second reverse convex surface such that said second reverse convex surface and said second stem are adapted to being mounted to a reconditioned humeral head associated with the humerus;
a first axis extending through a central location of said concave profile in said receiver and defining a non collinear angle relative to a second axis extending through a centerline of said second stem; and
said receiver further having a first interior component extending between an exposed surface of said concave profile which is intersected by a plane passing through said second annular perimeter, a dimension of said first interior component being greater than that of a second interior component extending beyond said first component to said second reverse convex bone contacting surface and in order to space the humerus from the scapula so as to maximize articulating motion between said ball and receiver.

2. The implant assembly as described in claim 1, each of said ball and receiver being constructed of at least one of a polymer, polymer composite, metal, metal composite or polymer/metal admixture.
